(19) United States
(12) Patent Application Publication
(10) Pub. No.: US 2021/0369892 A1
(43) Pub. Date: Dec. 2, 2021

(54) PORTABLE UV-C PATHOGEN INACTIVATION APPARATUS FOR HUMAN BREATHING AIR

(71) Applicant: MeadowStar Enterprises LTD, Goodyear, AZ (US)

(72) Inventors: Loren P. Hoboy, Avondale, AZ (US); Alexander J. Yarmie, Lovettsville, VA (US); Ronald T. Davis, Lancaster, CA (US)

(21) Appl. No.: 17/330,004

(22) Filed: May 25, 2021

Related U.S. Application Data
(60) Provisional application No. 63/032,503, filed on May 29, 2020.
(60) Provisional application No. 63/039,778, filed on Jun. 16, 2020.

(51) Int. Cl. A61L 2/10; A61L 2/00; A61M 1/02; A61M 1/36

(52) U.S. Cl. CPC A61L 2/10; A61L 2/0011; A61M 2205/053; A61M 1/3681; A61M 2202/0415; A61M 1/0281

(57) ABSTRACT

System and method for pathogen inactivation with UV-C light (employed by itself or in addition to filtering out particulates rom the flow of air reaching the user) by delivering, into a lightguide portion of the air inactivation chamber of the system, a dose of ultraviolet radiation sufficient for at least one log reduction level of the pathogen while, at the same time, multiply reflecting the light inside the chamber to increase the irradiance of inactivating light several fold (up to 5×, or even up to 8.6×) as compared to that delivered to the chamber.

### CROSS REFERENCE TO RELATED APPLICATIONS

This application claims priority from the U.S. Provisional Patent Applications Nos. 63/032,503 filed on May 29, 2020 and 63/039,778 filed on Jun. 16, 2020. The disclosure of each of the above-identified patent applications is incorporated herein by reference.

### TECHNICAL FIELD

The present invention relates generally to personal protective equipment (PPE) and, more particularly, to an apparatus configured to inactivate pathogens present in breathing air instead of attempting to filter-out such pathogens.

### RELATED ART

With the global pandemic of airborne pathogens such as COVID19, (SARS-CoV-2), and other calamities that will follow, there remains a persistent need in various PPE. Currently, filters devised to remove the particulate matter (interchangeably referred to herein as particulate filters) are being used to reduce risk of the spread of such diseases. These filters are rated pursuant to following Respirator Rating Number Class. The ones of class 95 (N95, which is designed to removes 95% of all particles that are at least 0.3 microns in diameter), class 99 (designed to remove 99% of particles that are at least 0.3 microns in diameter), and class 100 (P100, designed to remove 99.97% of all particles that are 0.3 microns in diameter or larger) are also known as HEPA Filters and are commonly used by the healthcare workers and first responders. However, most viruses are smaller in dimension than the mesh size of these particulate filters.

Droplets naturally produced by humans (e.g. droplets produced by breathing, talking, sneezing, coughing) contain various potentially infectious agents, e.g. bacteria, fungi and viruses in the mucous and saliva. As is well known in related art (see, for example, “The airborne lifetime of small speech droplets and their potential importance in SARS-CoV-2 transmission” by Valentyn Stadnytskyi, Christina E. Bax, Adriaan Bax, and Philip Anfinrud; PNAS first published May 13, 2020 https://doi.org/10.1073/pnas.200687411), “[s]peech droplets generated by asymptomatic carriers of severe acute respiratory syndrome coronavirus 2 (SARS-CoV-2) are increasingly considered to be a likely mode of disease transmission. Highly sensitive laser light scattering observations have revealed that loud speech can emit thousands of oral fluid droplets per second. In a closed, stagnant breathing air environment, they disappear from the window of view with time constants in the range of 8 to 14 min, which corresponds to droplet nuclei of ca. 4 μm diameter, or 12- to 21-μm droplets prior to dehydration. These observations confirm that there is a substantial probability that normal speaking causes airborne virus transmission in confined environments.” As these droplets carrying virus enter the breathing air, they begin to dehydrate. A logical conclusion is that as the droplets decrease in size due to the water in the mucus evaporating, and the droplet size continues to decrease rendering the particulate filter at least less effective (and probably substantially ineffective. This risk needs to be further mitigated.

It can be stated with substantial conviction, therefore, that the claimed operational performance of these particulate filters is not sufficiently compliant with removing near 100% (5-log or 6-log deactivation) of actual virus hazards, such as COVID 19. Indeed, viruses range in size from 0.02 to 0.5 Micron; bacteria range from 0.2 to 2 microns; spores range from 0.8 to 50 microns in size. SARS-CoV-2 (Covid 19) is a large-sized virus (of approximately 0.12 microns) while seasonal influenza A & B (flu) virus is approximately 0.17 microns. Particulate filters in common use alone cannot completely stop viruses or bacteria which are airborne, especially as the carrier droplets decrease in size due to dehydration when expelled into the air.

Moreover, according to the information presented in N, Engl. J. Med. 2020, March 17 by N. van Doremalen et al. “SARS-CoV-2 May remain Viable and Infectious for Hours in Aerosols . . . ”

Accordingly, there exists persistent need for more effective PPE to otherwise render viruses in breathing air ineffective, and specifically—improved respirator masks built to deactivate pathogens instead of just attempting to filter them out.

### SUMMARY

An embodiment of the present invention provides a pathogen inactivation apparatus that includes a closed hollow shell surrounding an inner volume inside the shell (here, a wall of the shell is fluidly impermeable). The embodiment also includes first and second fluid ports, at the shell, that have corresponding first and second axes. The fluid ports provide fluid connections between the inner volume and an outside of the shell. The embodiment additionally includes an optical port that is fluidly sealed at the shell and has a third axis and being configured to deliver target light in the UV-C spectral band from the outside of the shell towards the inner volume. The inner volume contains an optical element configured to recirculate the target light within the inner volume by redirecting a flux of the target light, received in the inner volume through the optical port, across the inner volume multiple times. In a specific case, such optical element may include an optical thin-film coating characterized by high reflectivity at a wavelength of the target light. (Such coating is present at least on portions of an inner surface of the wall that are transverse to the third axis). Additionally or in the alternative, the first and third axes may be substantially transverse to one another, while the embodiment includes comprising first and second baffles affixed to an inner surface of the wall to extend along the second axis such as to block a direct flow of fluid, received in the inner volume from the at least on fluid port, along the first axis and to redirect this flow into a channel formed between the first and second baffles and extending along the third axis. (in the specific implementation of the latter case, at least one of the following conditions may be satisfied:—the third axis may be defined to traverse the channel without crossing either of the first and second baffles; and—the second and third axes may be substantially transverse to one another. (If an when the inner surface of the wall is defined by a substantially cylindrical surface coated with a thin-film coating with high reflectivity at a wavelength of the target light, the inner surface with the coating is structured to reflect the target light along the channel between the first and second baffles.) In at least one implementation, the apparatus may be configured such as to satisfy at least one of the following conditions: i) at least one of the first and second baffles is substantially opaque at an operational wavelength of the target light, ii) at least one of the first and second baffles is highly-reflective at the operational wavelength; iii) the apparatus includes a beam-shaping optics affixed to the wall inside the inner volume an juxtaposed against the optical port; and/or to further comprise a facemask having a mask input port either directly or through a tubular member physically and fluidly connected with the second fluid port of the shell. Alternatively or in addition, the inner volume may be sub-divided to define a first sub-volume, a second sub-volume, and a third sub-volume (where the first and second sub-volumes are fluidly connected with one another only through a first gap formed in a first fluidly-impermeable partition extended across the inner volume along the third axis or between the first partition and the wall, and where the second and third sub-volumes are fluidly connected with one another only through a second gap formed in a second fluidly-impermeable partition extended across the inner volume along the third axis or between the second partition and the wall.) In such latter case, the first gap may be formed in a first portion of the inner volume that adjoins the optical port and the second gap is located in a second portion of the inner volume that is opposite to the optical port to define a path of fluid, received by the inner volume from the first fluid port and propagating towards the second fluid port, to extend along the third axis in the second sub-volume to maximize an overlap with a flux of target light received by the inner volume through the optical port. Alternatively or in addition, the apparatus may be configured to deliver an infrared (IR) irradiation into the inner volume to reduce moisture content from fluid entering the inner volume through one of the first and second fluid ports.

Embodiments of the invention additionally provide a method for operating a pathogen deactivation apparatus. The method includes the step of: transmitting air from the outside of the shell through the first fluid port into a first sub-volume of the inner volume, the first sub-volume being separated from a second sub-volume of the inner volume with a first substantially fluidly-impermeable screen that is configured to extend along the third axis and to define a first gap either in the first screen or between said first screen and the wall. The method additionally includes the step of passing the air along the third axis between said first screen and a second substantially fluidly-impenetrable screen that separates the second sub-volume of the inner volume from a third sub-volume of the inner volume, where the second screed is configured to extend along the third axis and to define a second gap either in the second screen or between said second screen and the wall. The method further includes the step of irradiating the air during the process of passing of the air through the second sub-volume with the target light (that has been delivered through the optical port into the second sub-volume) while recirculating said target light inside the second sub-volume by multiply reflecting such delivered target light at an optical member disposed in the inner volume to form inactivated air. In at least one case, the method also includes moving the air, that has passed through the second gap, through the second fluid port and through a facemask fluidly cooperated with the second fluid port, and/or (upon utilizing the air by a user wearing the facemask) expelling used air through the second fluid port to transmit said used air through the second gap while irradiating such used air with the target light being multiply reflected within the second sub-volume to form inactivated used air. The method may further include a step of irradiating the air during passing of said air through the inner volume to reduce a level of moisture in said air.

### BRIEF DESCRIPTION OF THE DRAWINGS

The invention will be more fully understood by referring to the following Detailed Description of Specific Embodiments in conjunction with the not-to scale Drawings, of which:

FIGS. 1A, 1B illustrate related embodiments of the apparatus configured, according to the idea of the invention, to include a sterilization unit or sub-system, a respiratory mask, and various peripheral components.

FIGS. 2, 3, and 4 present schematics of related and not-mutually exclusive implementations of the sterilization unit.

FIGS. 5, 6, and 7 illustrates versions of cooperation between at least one sterilization unit and a user-worn respiratory mask.

FIGS. 8, 9, 10 provide additional depictions of combinations of the sterilization unit of the embodiment of the invention with a user-worn mask.

FIGS. 11, 12 summarize data of calculations demonstrating the efficiency of pathogen-inactivating light recycling inside the chamber of an embodiment of the apparatus of the invention.

Generally, the size and relative scales of elements in drawings may be set to be different from actual ones to appropriately facilitate simplicity, clarity, and understanding of the drawings. For the same reason, not all elements present in one drawing may be necessarily be shown in another. Like elements and components are generally indicated/denoted with like numerals and labels.

### DETAILED DESCRIPTION

Table 1 below presents the data from “The size and duration of air-carriage of respiratory droplets and droplet-nuclei” (in J. of Hygiene 4:471-480 1946) that have been known for several decades:

TABLE 1

| Diameter Range | Number of Particles | Number of Particles |
|---|---|---|
| (microns) | in a Cough | in a Sneeze |
| 1-2 | 50 | 26000 |
| 2-4 | 290 | 160000 |
| 4-8 | 970 | 350000 |
| 8-16 | 1600 | 280000 |
| 16-24 | 870 | 97000 |
| 24-32 | 420 | 37000 |
| 32-40 | 240 | 17000 |
| 40-50 | 110 | 9000 |
| 50-75 | 140 | 10000 |
| 75-100 | 85 | 4500 |
| 100-125 | 48 | 2500 |
| 125-150 | 38 | 2500 |
| 150-200 | 35 | 1800 |
| 200-250 | 29 | 2000 |
| 250-500 | 34 | 1400 |
| 500-1000 | 12 | 2100 |
| 1000-2000 | 2 | 1000 |

A skilled person will readily appreciate that, while protective masks that the general population is asked to wear, may somewhat reduce the risk of catching a virus from a diseased person spreading such virus via airborne mucus droplets during breathing, talking, sneezing, or coughing, it is appreciated that for a healthy person, these facemask are potentially substantially ineffective in preventing contraction of airborne COVID 19, which is reported to remain active in the air for hours.

Embodiments of the invention solve the problem of rendering the air safe by inactivating the air-carried pathogens with light having a wavelength from the UV-C portion of the electromagnetic spectrum (from about 190 nm to about 280 nm) instead of or in addition to filtering the target pathogens our by delivering a sufficient (for de-activation characterized by at least one log reduction level, and in a preferred case—up to three to six log reduction levels) dose of ultraviolet radiation into the air passing through a hollow lightguide of an inactivation chamber of the proposed apparatus. Embodiments of the invention manifest in a sterilization apparatus for sterilizing air while connected with a full-face or half-face mask respirator worn by a human and address either or both the inactivation of human intake breathing air and the inactivation of breathing air expelled by a human.

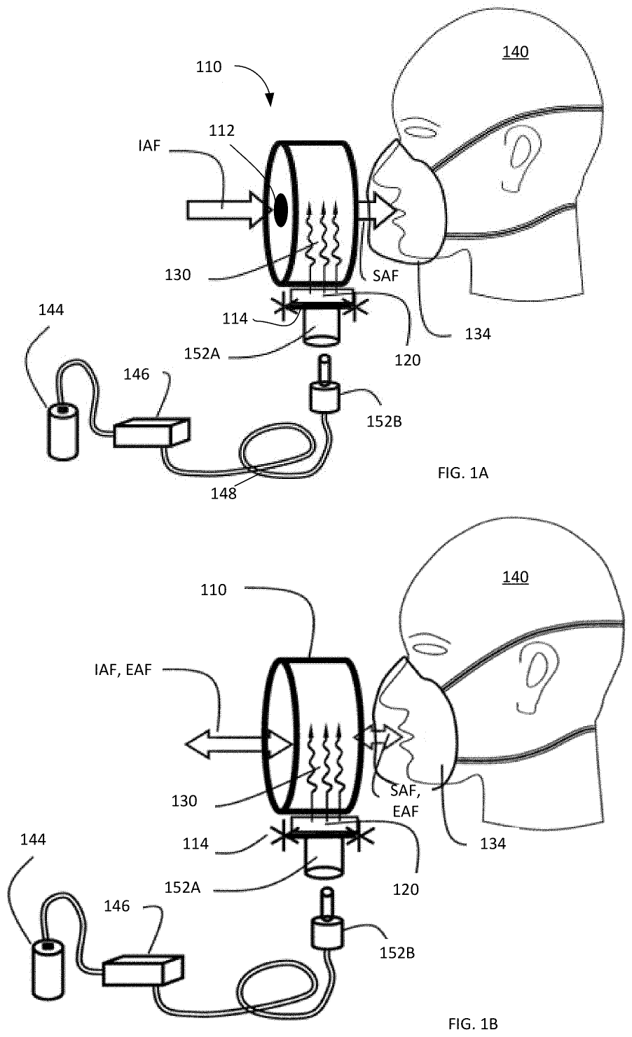

FIG. 1A provides a schematic of an embodiment 100 of the air-sterilization apparatus, showing, in general, a sterilization unit 110, defining a fluidly sealed inner chamber or cavity (which may contain air baffles extending between the opposite sides of the chamber, as discussed below) and having an inlet port 112 through which an intake air flow (shown as IAF) can be received by the unit 110. The source 120 of UV-light (preferably juxtaposed with the heatsink 124 for removal of waste heat from the UV emitter of the source 12) is appended to a side of the sterilization unit 110 and radiatively connected with the inner volume (chamber) of the unit 110 via an appropriate optical port (not shown) to irradiate (130) the inside of the inner chamber with a virus-inactivating radiation and with it—the flow of air A passing through the unit 110. The axes of the inlet port 112 and of the optical port are substantially transverse to one another. The sterilized air SAF is fluidly passed through the mask 134 (or another face-mouth-and-nose sealing contraption), to which the apparatus is affixed, and then to the user 140. The air sterilization apparatus 100 may be connected directly to an air intake valve of the mask 134 or to a hose that is, in turn, connected to such valve. The UV emitter(s) of the source 120 operate(s) at a wavelength within the range from about 190 nm to about 280 nm (that is, in a UV-C spectral range) and is/are preferably driven by a constant current from a power source (such as a battery and/or power electronic circuitry) 144, 146 connected through the power wire or cable 148 and the appropriate connector 152. Exhaust air exits through a separate exhaust valve, not shown.

A practically-reasonable modification to the above-outlined concept is to ensure that the air exhaled by the use of the embodiment of the invention is also sterilized. To this end, FIG. 1B illustrates a bi-directional version of the virus inactivation system of FIG. 1A. In this configuration the mask 134 has no exhaust valve and the normal inlet valve 112 can be removed to reduce breathing resistance due to pressure drop through the valves. The air flow exhaust EAF is delivered from the user 140 through the unit 110 and back to the environment.

UV-C light sources can include, without limitation, those provided by Klaran Crystal IS Green Island N.Y. USA, Bolb, Inc Livermore Calif. USA, Seoul Viosys, Gyeonggi-do Republic of Korea, Shenzhen Guangmai Electronics Co. Ltd. Shenzhen Guangdong China, Luminus Devices Inc 1145 Sonora Ct. Sunnyvale, Calif. 94086, as known in the art.

In a preferred embodiment, where the air sterilization apparatus 100 is portable, the battery and/or electronic circuitry 144, 16 can be carried in a pocket of the user 140, or be attached to the users belt, carried using a shoulder strap, purse, bag, backpack etc. The disconnection of the power jack 152A, 152B can be sued for turning the apparatus on/off. As an alternative, a power switch (not shown) can be installed in the circuit 146. Alternatively, power can be supplied directly from an AC power source through an AC/DC converter connected to the constant current producing electronics 146 if the uses is stationary and AC power is available.

It is appreciated that, in at least one case, the outer shell of the chamber 200 and/or baffles 216 are structured in such a fashion as to substantially reduce (by at least a coefficient of 100, preferably by a coefficient of 1,000) or even substantially completely prevent the target light, circulating inside the chamber 200 from escaping outside—for obvious reasons of increasing the safety of use of an embodiment of the invention.

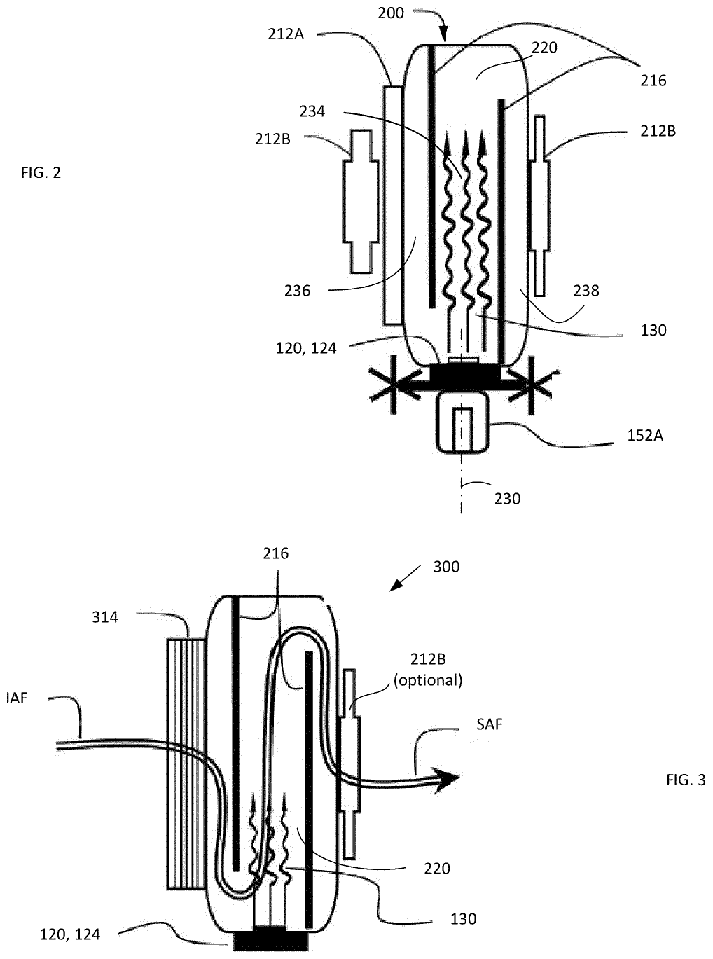

FIG. 2 depicts an embodiment 200 of the air sterilization unit 110 of FIG. 1, showing the inlet port 112 with an air intake grill 212A and/or air intake hose connection 212B. The inner chamber 220 of the unit 200 is preferably equipped with contains one or more air flow baffles 216 that are positioned and dimensioned to direct (uniformly channel) the incoming air through the space of the chamber 220 irradiated (130) with UV-light from the source of UV radiation 120 and to minimize the shortcircuiting.

The source 120 preferably includes an UV-C light source such as, without limitation, one of those provided by Klaran Crystal IS Green Island N.Y. USA, Bolb, Inc Livermore Calif. USA, Seoul Viosys, Gyeonggi-do Republic of Korea, Shenzhen Guangmai Electronics Co. Ltd. Shenzhen Guangdong China, as known in the art. In a preferred embodiment, the source 120 is configured to contain at least one UV-C LED emitting light at wavelength(s) within the range of 250 nm . . . 290 nm with the radiative power of about 30 mW to 100 mW.

In is preferred that the baffles and at least some of interior surface(s) of the disinfection chamber 220 may be configured to carry a highly-reflective coating(s) and/or be fabricated from the group of materials that are highly reflective in the selected UV range, in order to enhance disinfection efficacy by recycling UV light back and forth through the space 220 due to multiple reflections.

The inner surface of the chamber 220 of the unit 200 may be configured as a substantially cylindrical surface coated with a thin-film coating characterized by high reflectivity at a UV-C wavelength to reflect UV-C light delivered to the chamber through the optical port along the channel or lightguide formed between the first and second baffles. For example, the inside surface of the hollow chamber 220 can be enhanced (to possess the reflectivity of at least 50 percent and preferably at least 80 percent and even more preferably at least 96 percent) with at least one material from the group of high spectral or diffuse reflectance materials and coatings comprising: aluminum, protected polished aluminum, coaterd with a set of dielectric layers of UV enhanced aluminum, Alzak coated aluminum, UV Optical PTFE, UV, barium sulfate coating, zinc oxide, magnesium oxide, nitrocellulose filter paper, Tyvek, and aluminum foil, aluminized polyester, GORE DRP Diffuse Reflector Material, sintered PTFE lining, from the group of parts comprising Porex Virtek model PMR15, and GORE DRP Diffuse Reflector Material, and Avian-B.

The baffles 216 can be affixed to an inner surface of the inner chamber of the unit 200 to extend along the axis 230 of the optical port (through which the source(s) 120 are radiatively coupled with the hollow of the unit 200) to block a direct flow of fluid/air, received in the inner volume from the at least on fluid port (such as an inlet port 112 of FIG. 1A) of the unit 200 along the axis of such fluid port and to redirect this flow of fluid/air into a channel 234 formed between the first and second baffles 216 and extending along the axis 230. In one preferred case, the baffles are fabricated from Porex Virtek PTFE (Porex Corporation Fairburn, Ga.) with diffuse reflectance at the target UV-C wavelength of approximately 97%. In another implementation, the surfaces of the baffles 216 that are facing the axis 230 can be coated with a highly-reflective—at a wavelength of the source 120—coating.

With respect to how the baffles are dimensioned inside the chamber 220, the person of skill will understand that the inner volume 220 includes—when the baffles 216 are introduced—first, second, and third sub-volumes (236, 234, and 238, respectively). The first and second sub-volumes are fluidly connected with one another only through a first gap formed in either a first fluidly-impermeable partition (represented by one of the baffles 216 extended across the space 220 along the axis 230) or between such first partition and the inner wall of the unit 200. The second and third sub-volumes are fluidly connected with one another only through a second gap formed either in a second fluidly-impermeable partition (that is represented by another of the baffles 216) extended across the inner volume 220 along the axis 230 or between such second partition and the inner wall of the unit 200.

The first gap may be formed in a first portion of the inner volume 220 that adjoins the optical port of the embodiment and the second gap may be located in a second portion of the inner volume that is opposite to the optical port, to define a path of air, received by the inner volume 220 from the inlet port (such as port 112) and propagating towards the second fluid port of the unit (opposite to the inlet port) such as to extend this path along the axis 230 in the second sub-volume to maximize an overlap with a flux of pathogen-inactivating light received by the chamber 220 through the optical port.

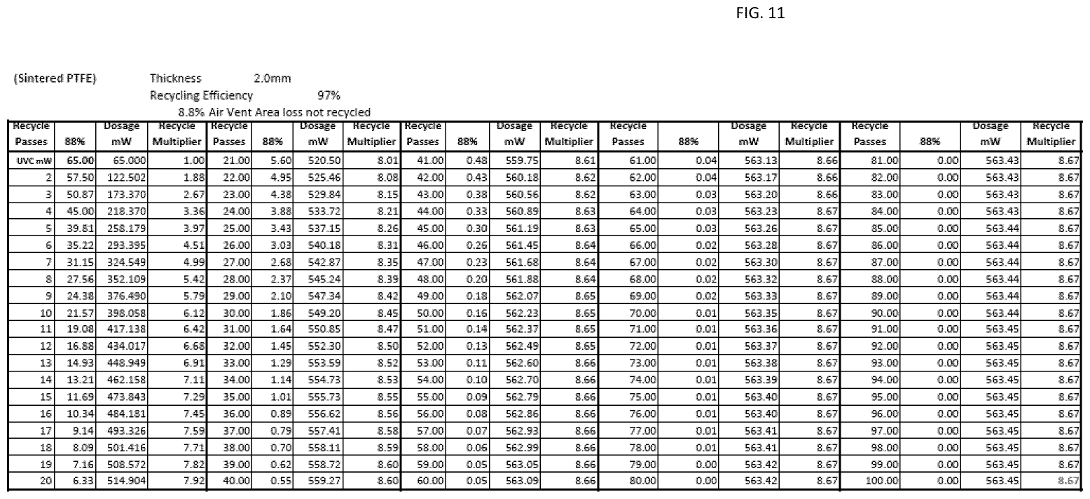

As an example of an alternative bur related and not mutually-exclusive arrangement, in at least one implementation, the inner volume of the chamber 220 may be complemented with an approximately 2-mm-thick sintered polytetrafluoroethylene (PTFE) generally cylindrical body (not shown) that is dimensioned as a hollow disk (that is, a closed hollow body having a cylindrical side surface and two base surfaces at the opposite sides of the cylindrical side surface). Such hollow body defines approximately 100 ml in volume, with an about 80 mm diameter and an about 20 mm height. The so-structured sintered PFTE body is molded into the chamber 220, assembled inside of the wall of the inner chamber 220, or otherwise juxtaposed with an internal surface of the wall of the inner chamber 220. (In a way, the hollow-disk-shaped PFTE body is configured as an inner lining of the chamber 200.) Each of the base surfaces has a corresponding opening or passage (an inlet vent and an outlet vent, respectively) defining a fluid path through the hollow disk, and the cylindrical side surface contains an opening dimensioned to accommodate the optical port of the embodiment 200. In this case, the base surfaces of the sintered PFTE hollow-disk-dimensioned body, that has been installed in the chamber 220, are configured to operate as baffles 216 while both the cylindrical side wall of such body and the base surfaces of it facilitate multiple-reflections of the target light, delivered through the optical port into the embodiment 200. Then, upon the proper assembly, the influx of air (IAF) enters the chamber 220 constructed of any suitable metal or plastic material through the inlet port of the embodiment 200, and then proceeds through an inlet vent on one end of the hollow-disk-shaped PFTE chamber (with the area of the inlet vent being approximately 4.4% of the area of the base surface of the hollow-disk body). The air exits on the opposite side of the inner chamber through a like-sized outlet vent. The inlet vent and the outlet vent are formed on the sides of the base surfaces that are opposite to one another with respect to the axis of the hollow-disk-shaped PFTE body. (In one specific case, when the volume inside the hollow-disk-shaped PFTE body is additionally sub-divided by a panel (˜80 mm by 0.5 mm) containing yet another opening therethrough, the air passing from the inlet port of the chamber 200 through the hollow-disk-shaped PFTE body first traverses the first half of the approximately 100 ml volume in the first direction, then passes through the opening in the panel, and then traverses the second half of the approximately 100 ml volume in a second direction that is substantially opposite to the first direction. The opening through the panel may also be dimensioned to have area of about 4.4% of the area of the based surface of the hollow-disk body. A skilled person will appreciate that in such a specific case, the air flow is forced to travel from one side of the chamber 220 to another side of the chamber 220 at least twice, thereby further increasing the length and/or volume of interaction of pathogen-inactivating target light with the air flow.) A Luminus XBT-3535-UV LED light source, delivering in operation about 65 mW of a 275 nm UV-C LED light is applied to the inner chamber 200 through the optical port thereof such that the UV-C light is split at the panel of the hollow-disk body to enter each half of it substantially in equal amount. The UV-C LED may be powered with a 12 volt Lithium Ion Batter and the current is controlled with a Meanwell LDD-600LW Series MEAN WELL Step-Down Mode CC DC-DC LED Driver. The described implementation results in deactivation of over 99.9% of a pathogen such as COVIS at normal human breathing rates (see FIG. 11 and Tables 2, 3 presented below).

Table 2 provides a summary of estimates of the UV-C light dosage required for indicated level of inactivation of a virus. The estimates take into consideration the publicly-available test results reporting that a 256 nm UV-C radiant flux with irradiance of 17 nW/m³ inactivates a virus in air at a 1-log level; these data is then used to calculates the equivalently-performing radiant flux at wavelengths of 222 nm and 275 nm (based on the equivalent amount of energy required to achieve the same inactivation level). This calculation methodology does not take into account that water has its highest radiant transmission at 270 nm, which suggests that when the virus containing airborne water droplets is being treated with UV-C light inactivation, a preferred embodiment wavelength selection should include light at approximately 270 nm for optimal inactivation efficacy.

TABLE 2

| | | Radiant flux | Radiant flux | Radiant flux |
|---|---|---|---|---|
| | | (mW/M3) | (mW/M3) | (mW/M3) |
| | | @256 nm | @222 nm | @275 nm |
| Energy J 10-19 | | 7.76 | 8.95 | 7.72 |
| 1-log reduction | 90.0000% | 17* | 14.7 | 18.3 |
| 2-log reduction | 99.0000% | 34 | 29.5 | 36.5 |
| 3-log reduction | 99.9000% | 51 | 44.2 | 54.8 |
| 4-log reduction | 99.9900% | 68 | 59.0 | 73.1 |
| 5-log reduction | 99.9990% | 85 | 73.7 | 91.4 |
| 6-log reduction | 99.9999% | 102 | 88.4 | 109.6 |

Table 3 contains information providing estimates of the required UV-V dosage

| | 90% Virus | 99% Virus | 99.9% Virus |
|---|---|---|---|
| | Inactivation | Inactivation | Inactivation |
| Outcomes | (mJ/cm2) | (mJ/cm2) | (mJ/cm2) |
| HCoV-229E | 0.56 | 1.1 | 1.7 |
| HCoV-OC43 | 0.39 | 0.78 | 1.2 |
| Influenza A (H1N1) | 1.3 | 2.6 | 3.8 |

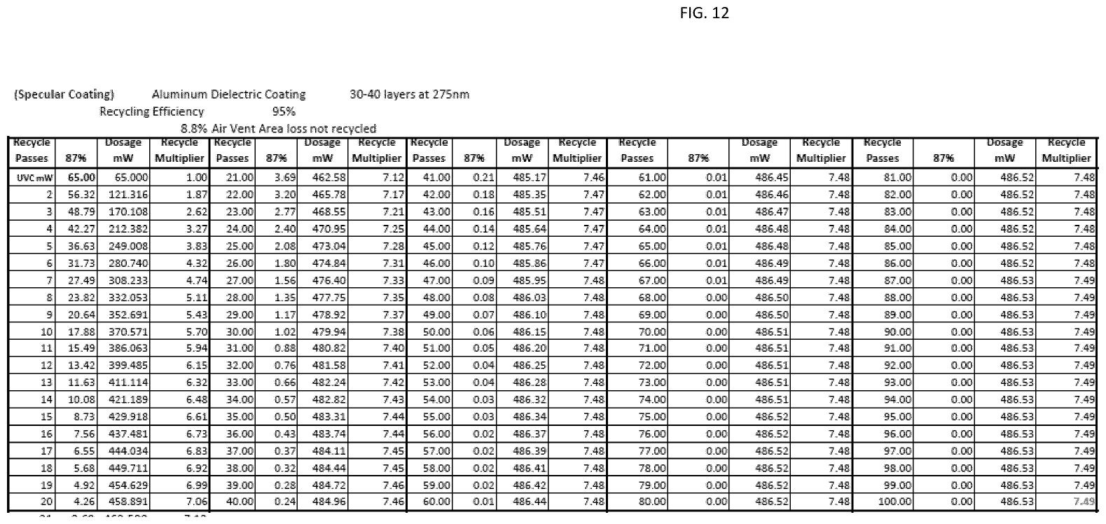

FIG. 11 contains a table presenting the results of calculation of efficiency of recycling of target light delivered to the embodiment of the inner chamber of the pathogen inactivation apparatus for an embodiment containing sintered as a light-reflecting PTFE material (discussed above). The data of FIG. 11 show the increase of light dose inside the chamber during the time required for an identified number of “reflections” (recycle passes) of occur (while the target light with radiative power of 65 mW is continually delivered to the chamber through the optical port). FIG. 12 is a table presenting the results of calculation of efficiency of recycling of target light delivered to the embodiment of the inner chamber of the pathogen inactivation apparatus for an embodiment containing an Al thin-film coating as a light-reflecting material. FIG. 12 showing the increase of light dose inside the chamber during the time required for an identified number of “reflections” (recycle passes) of occur (while the target light with radiative power of 65 mW is continually delivered to the chamber through the optical port).

FIG. 3 illustrates a related but not-mutually-exclusive implementation 300 of the sterilization apparatus 110, in which the air inlet or port 112 may contain, in addition to the elements 212A, 212B (not shown) the commercially-available particular filter (shown as 314). The path of the air flow from IAF at the inlet 212 through the inlet port 112, the disinfection inner chamber of the unit 300 and, finally, through the outlet of the unit 300 (SAF) toward the mask 134 and/or a hose connected to such mask.

The operational and dimensional characteristics of other constituent components and elements of the embodiment 300 may be chosen to be substantially the same as those discussed in reference to FIG. 2. The commercially available particulate filter 314 may be chosen to include N95 or P100, and HEPA filters for pathogen filtration. Such commercially available mask manufacturers selected from the group comprising 3M Company, St. Paul, Minn.; Mine Safety Appliances (MSA; Honeywell International Inc, Charlotte, N.C.; Jayco Safety Products Pvt. Ltd., Borivali, Mumnai, India; Draegerwerk AG & Co. KGaA, Lubeck, Germany; Gentex Corp, Zeeland, Mich., for example.

Depending on the specifics of design of filter 314, the air grill 212A may not be required, but the presence of the hose connector 212B may still be needed for ventilators, breathers, and CPAP machines. Information presented in FIGS. 11, 12 and Tables 2 and 3 is equally applicable to the operation of the embodiment 300.

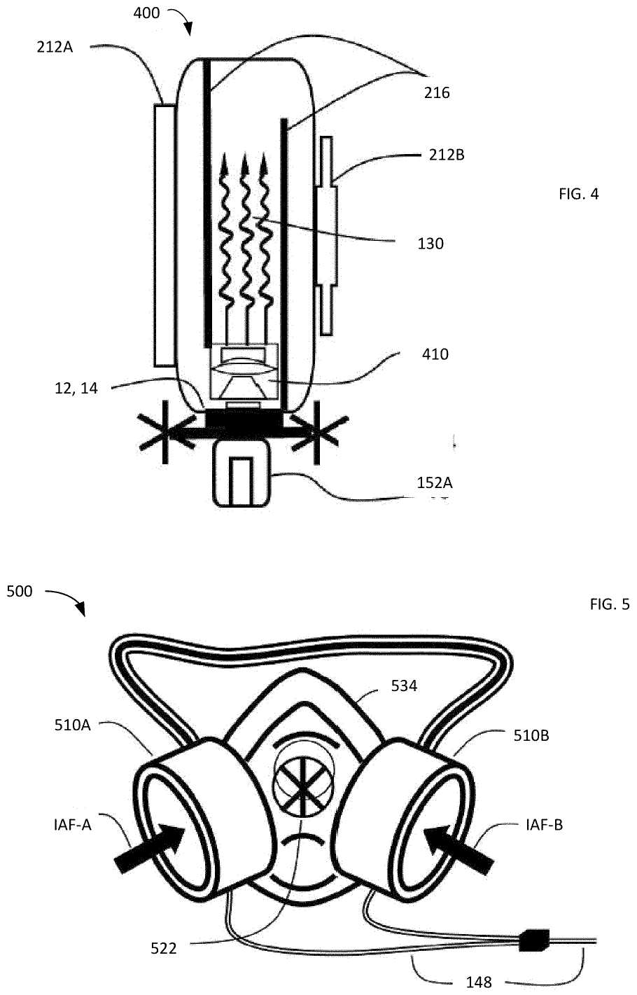

FIG. 4 illustrates a related embodiment 400 of the sterilization unit 110, in which a train 410 of judiciously-chosen optical components is employed to distribute the UV radiation 224 emanating from the source 120 more uniformly with in the treatment zone 220 of the disinfection air chamber. This optical train 410 may comprise collimation optics, cylindrical lens, structured LED linear radiation, collimating freeform and Fresnel line optics, fan angle line generator, line generation optics (such as a cylindrical lens), and/or retroreflector optics (in the simplest case—a mirror) designed to spatially shape the UV light (with the example of such design provided, for example, in U.S. Pat. No. 8,388,190 the disclosure of which is incorporated herein by reference) as will be apparent to those with skill in the art of optical design.

In one embodiment the optical train may include is a plano-convex cylindrical fused silica lens with a focal range from about 5 mm to 25 mm, and located at that focal length distance from the surface of the emitter of the light source 120. The operational and dimensional characteristics of other constituent components and elements of the embodiment 400 may be chosen to be substantially the same as those discussed in reference to FIG. 2. Information presented in FIGS. 11, 12 and Tables 2 and 3 is equally applicable to the operation of the embodiment 400.

It is appreciated that substantially every embodiment of the sterilization unit (such as embodiments 110, 200, 300, 400) includes inlet and outlet panels sealingly connected to one another with the use of a sidewall to form a hollow inner space/chamber/cavity therein. The inlet panel is configured to include an inlet for the air flow, the outlet panel includes an outlet for the air flow that has passed through the chamber and is structured to be juxtaposed with the mask 134 worn by the user. The air baffles that are separated from one another along an axis of the sterilization unit extend along the inlet panel and/or outlet panel between the portions of the sidewall to define a portion of the inner chamber that is judiciously irradiated with the virus sterilizing light delivered from the UV-light source(s) appended to the sidewall of the sterilization unit.

It is appreciated that while the general shape of the chamber of the sterilization units of the examples 100, 200, 300, 400 was described as cylindrical, in a related implementation the unit 100, 200, 300, 400 have be shaped differently—and have a pyramidal shape, for example (in a specific case—with a trapezoidal cross section as viewed along the axis of the inlet fluid port).

The skilled person now appreciates that, generally, an embodiment of a pathogen inactivation apparatus, structured according to the idea of the invention, includes a closed hollow shell surrounding an inner volume inside the shell (with a wall of the shell being substantially fluidly impermeable). The shell is complemented with first and second fluid ports that have corresponding first and second axes and providing fluid connections between the inner volume and an outside of the shell; and an optical port that is fluidly sealed at the shell. (The optical port has a third axis and is configured to deliver target light in the UV-C spectral band from the outside of the shell towards the inner volume. The inner volume contains an optical element configured to recirculate the target light within the inner volume by redirecting a flux of said target light, received in the inner volume through the optical port, across the inner volume multiple times and, in at least one specific case, to increase the irradiance of UV-C light circulating within the hollow chamber of the sterilization unit by at least 3×, preferably—at least 5×, and most preferably, by at least 8× as compared with the irradiance of pathogen-inactivating light delivered to the chamber from the source(s) 120.)

An embodiment of the air sterilization unit can be worn remotely, connected by a hose on a user's belt, or a lanyard around their neck. The inner chamber of the sterilization unit can be dimensioned to have any appropriate shape including, polyhedron, cylinder, cube, rectangular cuboid, cone, torus, and trapezoidal pyramid selected to promote mixed breathing air flow through the portion of the chamber occupied with the UV-C lightwaves when the source of UV light of the embodiment is activated.

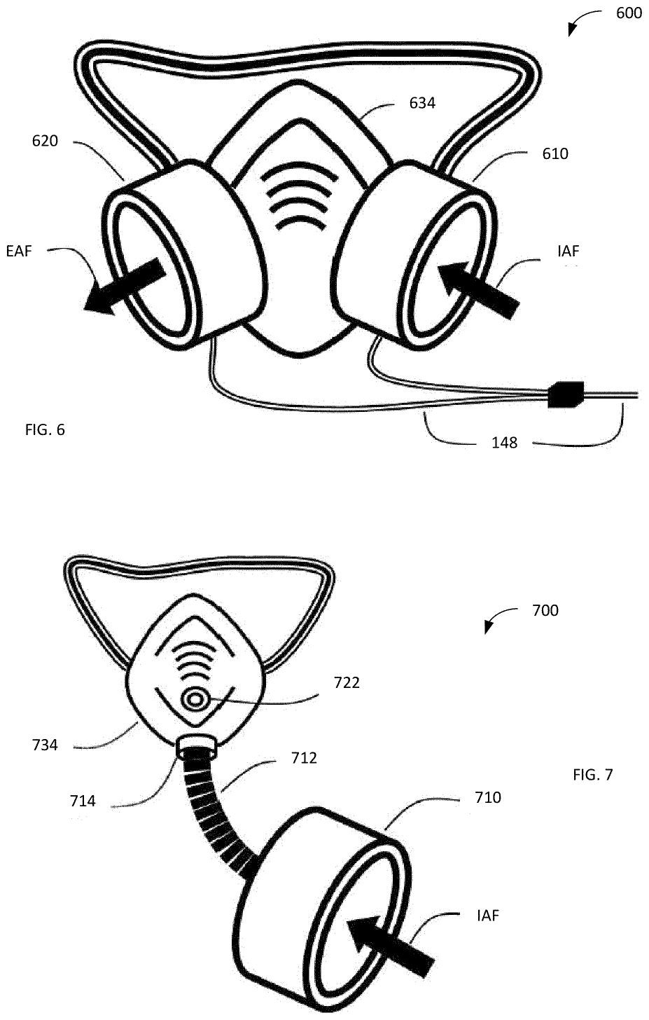

FIG. 5 schematically depicts an embodiment 500 of the sterilization apparatus for sterilizing breathing air in connection with full face or half face mask respirator 534. As shown, the embodiment 500 is equipped with multiple sterilization units 510A, 510B (shown for simplicity without the corresponding UV-light sources). Each of the sterilization units has a corresponding air-flow inlet port (indicated by the air-flows IAF-A, and IAF-B) and a corresponding air-flow outlet port (not seen; behind the units 510A, 510B as attached to the mask 534). The input air-flows IAF-A and IAF-B propagate through the chambers of the units 510A, 510B as discussed above and, upon being substantially sterilized, enter the mask 534 through the corresponding links. In this example, exhaust breathing air flow EAF is directed from inside the mask though the exhaust valve 522 without any additional treatment. Information presented in FIGS. 11, 12 and Tables 2 and 3 is equally applicable to the operation of the embodiment 500.

A related embodiment 600 of the apparatus is illustrated in FIG. 6. Here, the UV-C light irradiation is used for treatment of both the inhaled air flow IAF and exhaled air flow EAF. The first is carried out in the sterilization unit 510, delivering the sterilized air flow SAF to the user through the mask 634. The mask 634 is equipped with an appropriate outlet port (not shown) fluidly connecting the inner side of the mask with the sterilization unit 620. The exhaust breathing air EAF, which may be contaminated with pathogen in case the user of the device 600 is infected, is treated in the inner chamber of the second breathing air sterilization apparatus 620 before being discharged to the environment. The utilization of the sterilization apparatus system 600 has the potential to protect others from being infected by a diseased user especially in a crowded environment or contained space such as offices, hospitals, airplanes, stores, and even crowded entertainment venues. Information presented in FIGS. 11, 12 and Tables 2 and 3 is equally applicable to the operation of the embodiment 600.

FIG. 7 shows an embodiment 700, where the breathing air sterilization apparatus 710 system is deployed in conjunction with an breathing air flow hose 712 fluidly connecting the inner air inactivation chamber of the apparatus 710 and the air intake valve 714 of the user's mask 734. The exhaust valve 722 is configured to redirect the exhaust air outflow from the user wearing the embodiment 700 to the outside of the mask 734. Information presented in FIGS. 11, 12 and Tables 2 and 3 is equally applicable to the operation of the embodiment 700.

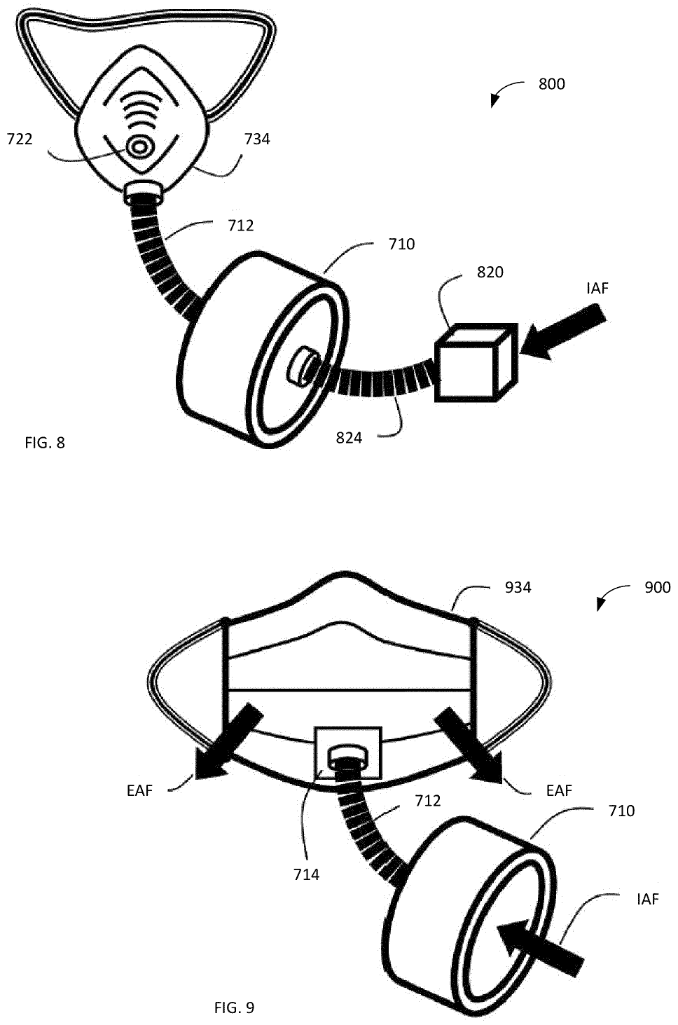

FIG. 8 depicts an embodiment 800 structured substantially similarly to the embodiment 700 but additionally equipped with the air-supply source 820 (which may be selected from an CPAP machine, fresh breathing air supply, and/or breathing ventilator) connected to the fluid inlet of the sterilization apparatus 710 with the use of a hose 824. Information presented in FIGS. 11, 12 and Tables 2 and 3 is equally applicable to the operation of the embodiment 800.

FIG. 9 illustrates an implementation 900 that is similar to the embodiment 700 but is used in conjunction with the cloth mask 934 configured to direct the exhaust breathing air flow EAF through the cloth mask to remove potentially contaminated water droplets. This configuration takes advantage of the fact that the breathing air intake flow resistance is lower through the breathing air sterilization chamber of the unit 710 than through the mask 934. It provides some protection to the general public against contamination by the user. Information presented in FIGS. 11, 12 and Tables 2 and 3 is equally applicable to the operation of the embodiment 900.

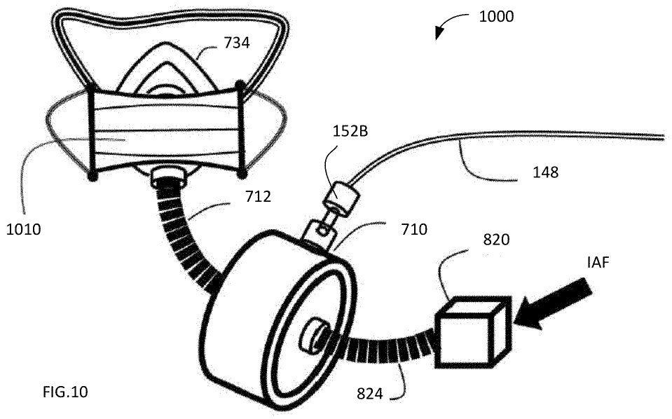

FIG. 10 illustrates a related implementation 1000 similar to that of FIGS. 7 and 8, but with the addition of a cloth filter mask 1010 over the exhaust valve 722 (not seen) to capture potentially pathogen laden water droplets from the exhale air, thereby reducing the risk of disease spread to the general public. Information presented in FIGS. 11, 12 and Tables 2 and 3 is equally applicable to the operation of the embodiment 1000.

Those with skill in the art, and encompassing the general principles defined here in will recognize the need to test the as designed and built subject Air Sterilization Apparatus System to determine that it meets the required efficacy for a specific pathogen and use.

Additionally or in the alternative—and as has been already alluded to above—reducing the size of pathogen-carrying water droplets in the breathing air stream can be assumed to reduce the dosage of UV-C required to inactivate the pathogen. In one embodiment, the optical source 112 (shown in FIG. 1A, for example) can include an IR LED selected to dehydrate the water droplets, facilitating UVC inactivation of the pathogen, with its radiation. In one case, such IR light source may be chosen to operate at a near IR wavelength (of, for example, about 1000 nm) at, for example, 850 mW of radiant power and is combined with an UV-C emitter as discussed above.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention. Within this specification, embodiments have been described in a way that enables a clear and concise specification to be written, but it is intended and will be appreciated that embodiments may be variously combined or separated without parting from the scope of the invention. In particular, it will be appreciated that all features described herein may be applicable to all aspects of the invention. Shown embodiments can be variably modified to better achieve goals of the invention.

When the present disclosure describes features of the invention with reference to corresponding drawings (in which like numbers represent the same or similar elements, wherever possible), the depicted structural elements are generally not to scale, and certain components are enlarged relative to the other components for purposes of emphasis and understanding. It is to be understood that no single drawing is intended or can be devised to support a complete description of all features of the invention. In other words, a given drawing is generally descriptive of only some, and generally not all, features of the invention. A given drawing and an associated portion of the disclosure containing a description referencing such drawing may not, generally, contain all elements of a particular view or all features that can be presented is this view, at least for purposes of simplifying the given drawing and discussion, and directing the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this particular detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

Moreover, if the schematic flow chart diagram is included, the depicted order and labeled steps of the logical flow are indicative of one embodiment of the presented method. Other steps and order of steps may be conceived that are equivalent in function, logic, or effect to one or more steps, or portions thereof, of the illustrated method. Without loss of generality, the order in which processing steps or particular methods occur may or may not strictly adhere to the order of the corresponding steps shown.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. The use of this term in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated may vary within a range defined by an experimental measurement error that is typical when using a measurement method accepted in the art for such purposes. For example, the terms “approximately” and about”, when used in reference to a numerical value, represent a range of plus or minus 20% with respect to the specified value, more preferably plus or minus 10%, even more preferably plus or minus 5%, most preferably plus or minus 2%.

The invention as recited in claims appended to this disclosure is intended to be assessed in light of the disclosure as a whole, including features disclosed in prior art to which reference is made. Furthermore, disclosed aspects, or portions of these aspects, may be combined in ways not listed above. Accordingly, the invention should not be viewed as being limited to the disclosed embodiment(s).

What is claimed is:

1. A pathogen inactivation apparatus comprising:
a closed hollow shell surrounding an inner volume inside the shell, a wall of the shell being fluidly impermeable;
first and second fluid ports, at said shell, the first and second ports having corresponding first and second axes and providing fluid connections between the inner volume and an outside of the shell; and
an optical port at said shell that is fluidly sealed at said shell, the optical port having a third axis and being configured to deliver target light in the UV-C spectral band from the outside of the shell towards the inner volume;
wherein the inner volume contains an optical element configured to recirculate the target light within the inner volume by redirecting said target light, received in the inner volume through the optical port, across the inner volume multiple times.

2. The apparatus according to claim 1, wherein said optical element is configured as an optical reflector at an operational wavelength of target light and disposed on an inner surface of the wall.

3. The apparatus according to claim 2, wherein said optical element includes at least one of
3a) an optical thin-film coating characterized by high reflectivity at a wavelength of the target light, said coating disposed at least on portions of an inner surface of said wall that are transverse to the third axis; and
3b) a layer of sintered polytetrafluoroethylene.

4. The apparatus according to claim 1,
wherein the first and third axes at substantially transverse to one another, and
further comprising first and second baffles affixed to an inner surface of the wall to extend along the second axis such as
to block a direct flow of fluid, received in the inner volume from the at least on fluid port, along the first axis and
to redirect said flow into a channel formed between the first and second baffles and extending along the third axis.

5. The apparatus according to claim 4, wherein at least one of the following conditions is satisfied:
5a) said third axis traverses the channel without crossing either of the first and second baffles; and
5b) the second and third axes are substantially transverse to one another.

6. The apparatus according to claim 4, wherein the inner surface of the wall is substantially a cylindrical surface coated with a thin-film coating characterized by high reflectivity at a wavelength of the target light, the inner surface with the coating configured to reflect said target light along the channel between the first and second baffles.

7. The apparatus according to claim 4, wherein at least one of the following conditions is satisfied:
7a) at least one of the first and second baffles is substantially opaque at an operational wavelength of the target light,
7b) at least one of the first and second baffles is highly-reflective at the operational wavelength;
7b) the apparatus includes a beam-shaping optics affixed to the wall inside the inner volume an juxtaposed against the optical port

8. The apparatus according to claim 4, comprising further comprising a facemask having a mask input port either directly or through a tubular member physically and fluidly connected with the second fluid port of the shell

9. The apparatus according to claim 1,
wherein the inner volume includes a first sub-volume, a second sub-volume, and a third sub-volume,
wherein the first and second sub-volumes are fluidly connected with one another only through a first gap formed in a first fluidly-impermeable partition extended across the inner volume along the third axis or between said first partition and the wall,
wherein the second and third sub-volumes are fluidly connected with one another only through a second gap formed in a second fluidly-impermeable partition extended across the inner volume along the third axis or between said second partition and the wall.

10. The apparatus according to claim 9, wherein the first gap is located in a first portion of the inner volume that adjoins the optical port and the second gap is located in a second portion of the inner volume that is opposite to the optical port to define a path of fluid, received by the inner volume from the first fluid port and propagating towards the second fluid port, to extend along the third axis in the second sub-volume to maximize an overlap with a flux of target light received by the inner volume through the optical port.

11. The apparatus according to claim 1, configured to deliver an infrared (IR) irradiation into the inner volume to reduce moisture content from fluid entering the inner volume through one of the first and second fluid ports.

12. A method for operating an apparatus that includes
a closed hollow shell surrounding an inner volume inside the shell, a wall of the shell being fluidly impermeable;
first and second fluid ports, at said shell, the first and second ports having corresponding first and second axes and providing fluid connections between the inner volume and an outside of the shell; and
an optical port at said shell that is fluidly sealed at said shell, the optical port having a third axis and being configured to deliver target light in the UV-C spectral band from the outside of the shell towards the inner volume;
the method comprising:
transmitting air from the outside of the shell through the first fluid port into a first sub-volume of the inner volume, the first sub-volume being separated from a second sub-volume of the inner volume with a first substantially fluidly-impermeable screen that is configured to extend along the third axis and to define a first gap either in said first screen or between said first screen and the wall;
passing said air along the third axis between said first screen and a second substantially fluidly-impenetrable screen that separates the second sub-volume of the inner volume from a third sub-volume of the inner volume, said second screed configured to extend along the third axis and to define a second gap either in said second screen or between said second screen and the wall, and
irradiating the air during said passing of the air through the second sub-volume with the target light delivered through the optical port into the second sub-volume while recirculating said target light inside the second sub-volume by multiply reflecting said target light at an optical member disposed in the inner volume to form inactivated air.

13. The method according to claim 12, wherein said multiply reflecting includes reflecting said target light at an optical reflector juxtaposed with an inner surface of the wall.

14. The method according to claim 12, wherein one of the first and second gaps is located next to the optical port while another of the first and second gaps is located next to a portion of the wall that is opposite to the optical port.

15. The method according to claim 12, further comprising
moving said air, that has passed through the second gap, through the second fluid port and through a facemask fluidly cooperated with the second fluid port.

16. The method according to claim 15, further comprising:
upon utilizing said air by a user wearing the facemask, expelling used air through the second fluid port to transmit said used air through the second gap while irradiating said used air with the target light being multiply reflected within the second sub-volume to form inactivated used air.

17. The method according to claim 16, further comprising: transmitting the inactivated used air through the first gap and through the first fluid port to the outside of the shell.

18. The method according to claim 12, further comprising: irradiating the air during passing of said air through the inner volume to reduce a level of moisture in said air.
